(19) United States
(12) Patent Application Publication
(10) Pub. No.: US 2012/0215127 A1
(43) Pub. Date: Aug. 23, 2012

(54) IN VIVO FLOW SENSOR

(75) Inventors: Mitsuhiro Shikida, Aichi (JP); Tsutomu Kawabe, Aichi (JP); Miyoko Matsushima, Aichi (JP); Takuo Yokota, Aichi (JP); Satoshi Iwai, Aichi (JP); Naoyuki Matsunaga, Aichi (JP)

(73) Assignee: National University Corporation Nagoya University, Aichi (JP)

(21) Appl. No.: 13/502,320

(22) PCT Filed: Aug. 10, 2010
(86) PCT No.: PCT/JP2010/063568

§ 371 Date: Apr. 16, 2012

(30) Foreign Application Priority Data
Oct. 14, 2009 (JP) 2009-237300

(51) Int. Cl. A61B 5/103; A61B 5/087

(52) U.S. Cl. 600/538; 600/587

(57) ABSTRACT

A flow sensor measures an amount of flow of a medium flowing inside a tubular organ of an organism by mounting a flexible substrate, upon which heaters are formed, inside a tube disposed in a manner so as to follow the tubular organ of the organism through which the medium is flowing, and by detecting a state of heat quantity generated from the heaters on the mounted flexible substrate being transmitted to the medium. In the flow sensor, at least the two heaters, formed upon the flexible substrate, respectively form symmetrical structures with identical heating capabilities under identical conditions, and the linear members, which supply power to the heaters from outside the organism, form symmetrical structures with identical heating capabilities under identical conditions, thereby allowing temperature distribution of the heaters and linear members inside the tube to he symmetrical. The plurality of linear members protrude in a manner so as to intersect from inside the tubular organ to outside the organism.

### TECHNICAL FIELD

The present invention relates to an in vivo flow sensor that can detect an amount of flow of a medium such as air, liquid, etc. flowing inside a tubular organ of an organism.

### BACKGROUND ART

Conventionally, there is a flow sensor configured to mount a flexible substrate, upon which a heater is formed, on an inner wall of a tube so that the flexible; substrate follows a shape of the inner wall of the tube. In the flow sensor, the sensor is formed on a film having a thickness of a few microns, and then mounted on an inner wall surface of the tube at which a flow rate becomes minimum. Thus, the flow sensor can reduce an increase of fluid resistance caused by sensor installation to the utmost limit (see Patent Document 1, for example). Also, there is a technique of reducing the size of the aforementioned flow sensor by means of a heat shrinkable tube (see Patent Document 2, for example).

Patent Document 1: Unexamined Japanese Patent Application Publication No. 2007-127538

Patent Document 2: Unexamined Japanese Patent Application Publication No. 2009-168480

### DISCLOSURE OF THE INVENTION

### Problems to be Solved by the Invention

Each of the flow sensors mentioned in Patent Documents 1 and 2 is configured to be assembled to a catheter structure. It is not possible to implant the Row sensor into a tubular organ of an organism to measure an amount of flow of a medium inside the tubular organ, e.g., it is not possible to implant the flow sensor into an air passage of a rat or the like for use in an animal study. Also, in the flow sensor of a conventional catheter structure, electric wiring to the sensor extends along a direction of flow of a fluid inside the tube. Therefore, depending on the direction of flow inside the tube, sensor output characteristics with respect to an amount of flow (an output value when the fluid flows from an upstream side and an output value when the fluid flows from a downstream side) differ. There is a problem in that a reciprocating current such as exhalation and inhalation cannot be measured accurately.

Details of such situation will be described below in Patent Document 1, it is described that “taking-out of an electrode to a heater is conducted by bending part of a flexible substrate, on which the heater is formed, at an end of a tube inside which a fluid flows toward outside the tube”. Also, according to the description of the drawings attached to the application of Patent Document 1, “thin film wiring connected to the heater is formed on the flexible substrate in the same manner as the heater, and the wiring is configured to be taken out from one direction of the tube”.

Therefore, in Patent Document 1, “the thin film wiring which supplies power to the heater is configured to be taken out firstly from one direction of the tube inside the tube and finally bent toward outside the tube at the end of the tube so as to be connected to outside”. As a result, the sensor output characteristics differ between the case in which the flow of the fluid inside the tube flows from the upstream side and the case in which the flow of the fluid inside the tube flows from the downstream side.

In Patent Document 2 as well, basically, “the thin film wiring which supplies power to the heater is configured to be taken out from one direction of the tube inside the tube”, similar to the case of the aforementioned Patent Document 1. Therefore, the same phenomenon

The reason why “a reciprocating current of a medium inside the tube cannot be measured accurately when the thin film wiring which supplies power to the heater is configured to be taken out from one direction of the tube inside the tube” was considered in detail. As a result,the following observation was made.

The thin film wiring which supplies power to the heater also has an electric resistance. Thus, the thin film wiring functions marginally the same as a heating element. Therefore, as the thin film wiring is taken out from the direction of flow of the fluid inside the tube, the thin film wiring also becomes a heating element. Thus, heat distribution on the heater becomes asymmetrical. As a result, sensor output characteristics vary depending on the direction of the fluid flowing inside the tube.

As a result, there is no problem if an amount of flow in one direction is merely measured. However, in the case of measurement of a reciprocating current such as exhalation and inhalation, sensor output values differ depending on the direction of flow of the fluid. A problem occurs in that an amount of flow of the fluid cannot be accurately measured.

The present invention has been made to solve the above problem. One object of the present invention is to provide an in vivo flow sensor that is implanted in a tubular organ of an organism and can accurately measure an amount of flow of a medium inside the tubular organ.

### Means to Solve the Problems

An in vivo flow sensor according to a first aspect of the present invention in order to achieve the above object measures an amount of flow of a medium flowing inside a tubular organ of an organism by mounting a flexible substrate, upon which heaters are formed, inside a tube that is disposed in a manner so as to follow the tubular organ of the organism inside which the medium is flowing, and by detecting a state of heat quantity generated from the heaters on the mounted flexible substrate being transmitted to the medium.

At least the two heaters formed on the flexible substrate respectively form symmetrical structures with identical heating capabilities under identical conditions. Also, linear members, which supply power to the respective heaters from outside the organism, form symmetrical structures with identical heating capabilities under identical conditions. Thereby, heat distribution by the heaters and the linear members inside the tube is made symmetrical.

The plurality of linear members protrude in a manner so as to intersect from inside the tubular organ to outside the organism.

In the above described configuration, the at least two heaters formed on the flexible substrate for flow measurement are configured to form symmetrical structures with identical heating capabilities under identical conditions. Also, the linear members, which supply power to the respective heaters from outside the organism, are configured to form symmetrical structures with identical heating capabilities under identical conditions. Thereby, heat distribution by the heaters and the linear members inside the tube is made symmetrical. Regardless of a direction of flow of the medium inside the tube, sensor output characteristics to flow of the medium flowing inside the tube can be the same. As a result, a reciprocating current such as exhalation and inhalation can be accurately measured. Also, if the flexible substrate is mounted on an inner wall of the in vivo tubular organ so as to follow the shape of the inner wall of the in vivo tubular organ, flow resistance accompanied by sensor installation can be reduced to the utmost limit.

Also, “a plurality of heaters with identical heating capabilities under identical condition” means, for example, a case in which the heaters are identical in size and shape, if the plurality of heaters are formed from the same material. If the heaters are formed from different materials, a case is also included in which the heaters are not identical in size and shape. Further, “linear members with identical heating capabilities under identical condition” means the same configuration as that of the above heaters.

The plurality of linear members protrude in such a manner as to intersect from inside the tubular organ to outside the organism. Thus, via the plurality of the linear members, electric power can be supplied to the heaters on the flexible substrate from outside the organism. Thereby, for example, even if the sensor is implanted in a tubular organ inside an organism such as a human and an animal, signals between the sensor and outside the organism can be transmitted/received. As the tubular organ inside an organism, there are, other than an air passage through which air flows as a medium, a blood vessel and a ureter through which liquid flows as a medium, and an intestine through which solid and fluid matters flow as media.

There are various methods of supplying electric power to the linear members from outside. For the purpose of supplying electric power to the plurality of linear members from outside by wire or wirelessly, it is preferable that the linear members may be configured to penetrate the tubular organ of the organism and protrude outside the organism.

In this manner, electric power can be supplied from outside the organism. Without limitation of capacity of a battery, flow of the medium inside the tubular organ of the organism can be measured over a long duration.

The “linear members to which electric power is supplied by wire” mean, for example, power source wiring of copper wire and aluminum wire coated so as not to negatively affect the organism as much as possible. The “linear members to which electric power is supplied wirelessly” mean, for example, linear or loop antennas.

The organism has operational sections such as arms, legs, a mouth and a tail. Thus, upon detection of an amount of flow of the medium inside the tubular organ, the linear members may be possibly destroyed by the operational sections. Therefore, as is the case in a second aspect of the present invention, it is preferable that portions of the linear members protruding outside the organism are at, positions where it is difficult for the operational sections of the organism to come into contact.

Further, if the portions protruding outside the organism of the linear member are at positions not to be destroyed h the operational portions of the organism, it is convenient since the linear members are not to be destroyed by the operational portions of the organism during measurement of the amount of flow of the medium inside the tubular organ.

Further, as is the case in a third aspect of the present invention, a position where the in vivo flow sensor is installed is in an air passage of an animal, and electric wiring members connected to the linear members are taken outside the animal from a back of the animal.

If the position where the in vivo flow sensor is installed is in air passage as such, it is preferable that the linear members are taken outside the animal in such a mariner as to protrude from the air passage, and the electric wiring members connected to the linear members are taken outside the animal from a back of the organism after drawn to the hack of the organism.

### BRIEF DESCRIPTION OF THE DRAWINGS

FIG. 1 is a diagram showing conceptionally a use of an implantable miniature flow sensor of the present embodiment.

FIG. 2 is a view showing a structure of the implantable miniature flow sensor of the present embodiment.

FIGS. 3A-3D are views showing connecting methods of thin film wirings of a film substrate and wirelike electric wirings used in the implantable miniature flow sensor of the present embodiment.

FIG. 4 shows a photo of a film flow sensor of the present embodiment.

FIGS. 5A-5F are views showing techniques of reducing the size of the implantable miniature flow sensor of the present embodiment to an implantable size of a diameter of a few millimeters or less.

FIG. 6 is a drawing showing a mounting state of the implantable miniature flow sensor of the present embodiment.

FIG. 7 is a diagram showing an example of a relationship between input power and a detected amount of flow of the flow sensor according to the present embodiment.

FIGS. 8A-8C are diagrams showing temperature distribution on heaters, and a relationship between an amount of flow and sensor output in case of an electric wiring taken-out structure in a conventional flow sensor (conventional sensor), as a comparative example.

FIGS. 9A-9C are diagrams showing temperature distribution on heaters, and a relationship between an amount of flow and sensor output in case of an electric wiring taken-out structure in the flow sensor of the present embodiment,

FIGS. 10A-10B are diagrams showing mounting states of the implantable miniature flow sensor of the present embodiment implanted to a rat as an experimental animal.

FIG. 11 is a diagram showing a result of evaluation of exhalation and inhalation characteristics at an air passage during activity of the rat directly by the implantable flow sensor of the present embodiment, after the flow sensor is implanted to the air passage of the rat.

### EXPLANATION OF REFERENCE NUMERALS

1 . . . experimental animal, 2 . . . air passage (tubular organ), 3 . . . implantable miniature flow sensor, 4 . . . wire-like electric wiring (electric wiring member), 5 . . . film flexible electric wiring, 10 . . . film flow sensor, 11 . . . film substrate, 12 . . . heater, 13 . . . thin film wiring (linear member), 15 . . . anisotropically conductive film, 20 . . . heat insulating cavity structure, 21 . . . flow passage, 30 . . . tube, 40 . . . positioning jig, 41 . . . groove, 42 . . . resin film, 50 . . . heat shrinkable tube, 51 . . . slit for taking out wiring.

### MODE FOR CARRYING OUT THE INVENTION

Hereinafter, the best mode to practice the present invention will be described in more detail.

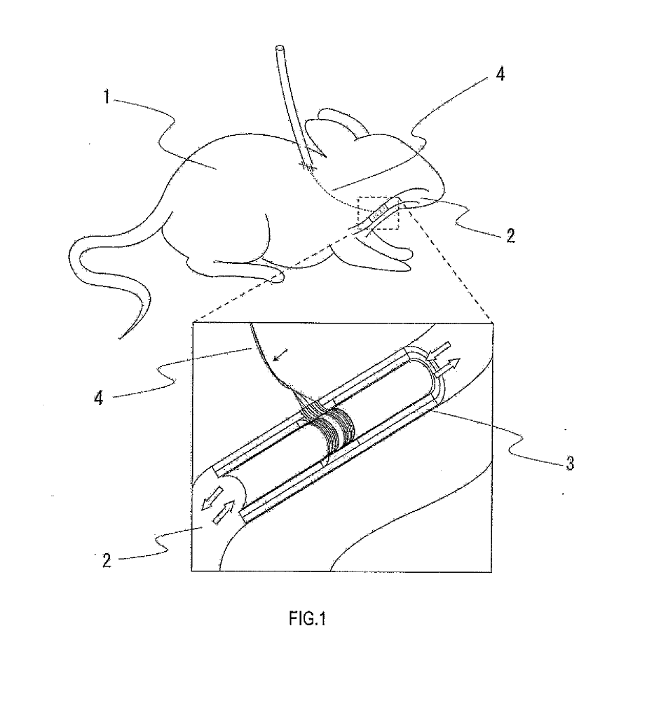

FIG. 1 shows conceptionally a use of an implantable miniature flow sensor of an embodiment according to the present invention. The implantable miniature flow sensor 3 is implanted in an air passage 2 of an experimental animal 1 such as a rat and a mouse. Thereafter, exhalation and inhalation during activity of the experimental animal 1 are quantitatively measured and evaluated by the implantable miniature flow sensor 3.

Wire-like electric wirings 4 are used for transmission of electric signals between the implantable miniature flow sensor 3 and the outside. The wire-like electric wirings 4 are connected with the implantable miniature flow sensor 3. The wire-like electric wirings 4 are extended to a back portion of the experimental animal under the skin, and taken outside at the back.

Thereby, upon measuring and evaluating exhalation and inhalation during activity of the experimental animal 1, the wire-like electric wirings 4 are prevented from being scratched by operational sections such as hands, legs, a mouth or a tail of the animal. Thus, the wire-like electric wirings 4 will not be destroyed by the operational sections such as hands of the animal.

It is preferable that an outer diameter of the implantable miniature flow sensor is determined in accordance with the size of the air passage 2 of the experimental animal for use. For example, it is preferable that the outer diameter of the implantable miniature flow sensor is around 1.5 mm to 1.8 mm in ease of a mouse., and around 1.8 mm to 2.0 mm in case of a rat.

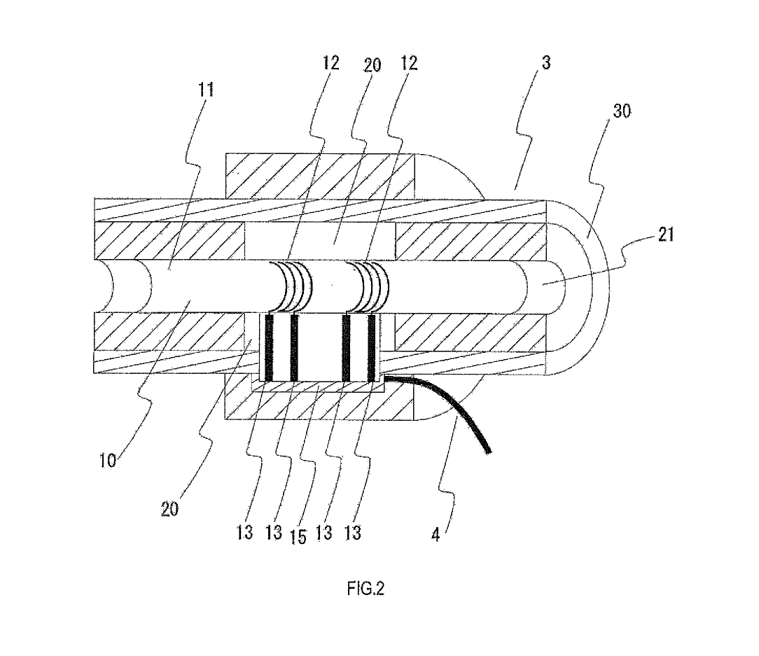

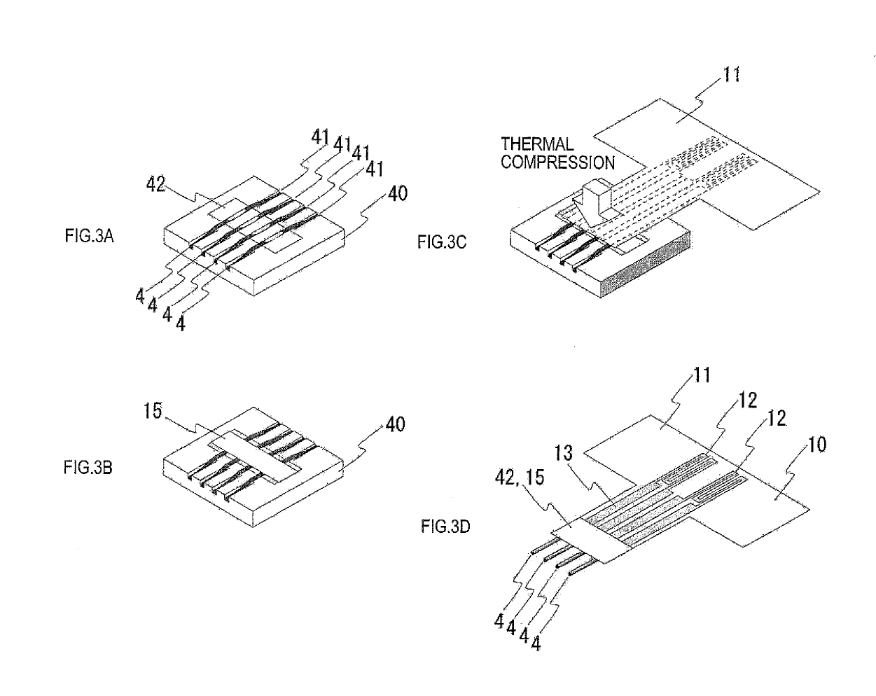

FIG. 2 shows a structure of the implantable miniature flow sensor of the embodiment according to the present invention.

The implantable miniature flow sensor 3 includes: a film flow sensor 10 arranged so as to follow an inner wall of a tube; a heat insulating cavity structure 20; a flow passage 21; and the tube 30. The film flow sensor 10 includes heaters 12 formed on a film substrate 11, and thin film wirings 13 that supply electric power to the heaters. The film substrate 11 is arranged so as to follow the inner wall of the tube 30. Thereby, turbulence in flow of fluid flowing through the flow passage 21 inside the tube 30, which is accompanied by installation of the sensor 10 inside the tube 30, is reduced to the ultimate limit.

Two pairs of the heaters 12 and the thin film wirings 13 formed on the film substrate 11 are identical in shapes, respectively, and form symmetrical structures with identical heating capabilities under identical conditions. Thereby, heat distribution by the heaters 12 and the thin film wirings 13 inside the tube 30 is configured to be symmetrical.

Further, the thin film wirings 13 are arranged to protrude from the air passage as a tubular organ to outside the organism. Particularly, the thin film wirings 13 are taken out from inside the tube 30 so as to intersect with (be perpendicular to) flow of the fluid flowing through the flow passage 21 inside the tube 30. Even if a direction of flow is reversed, the two pairs of the heaters 12 and the thin film wirings 12 are in a reversed state under identical conditions. Thereby, regardless of the direction of flow of the medium inside the tube 30, sensor output characteristics with respect to an amount of flow of the fluid are the same.

Also an anisotropically conductive film 15 is used to connect each of the wire-like electric wirings 4 with each end of the thin film wirings 13 provided on the film substrate 11. Thereby, even in a case where the implantable miniature flow sensor 3 is implanted in an air passage of an animal, the wire-like electric wirings 4 can be easily drawn to a back portion of the animal under the skin. Also, even after the wire-like electric wirings 4 are taken out from the back portion, wirings can be taken out with high reliability.

It is preferable to determine a diameter to be employed, in consideration that physical tension may be applied to the wire-like electric wirings 4. The thin film wirings 13 connected to the wire-like electric wirings 4 (electric wiring members) via the anisotropically conductive film 15 function as linear members that supply electric power to the heaters 12 of the implantable miniature flow sensor 3.

In the implantable miniature flow sensor 3, a heat insulating cavity structure 20 is formed at an outer peripheral portion of the heaters in order to achieve heat insulation of the heaters 12 formed on the film substrate 11. For the purpose of forming the heat insulating cavity structure 20, the tube 30 of the implantable miniature flow sensor 3 has a double structure of resin material. Thereby, heat insulation to the heater substrate is achieved. Aspiration of ten to twenty times per minute can be measured.

At a portion connecting the thin film wirings 12 and the wire-like electric wirings 4 via the anisotropically conductive film 15, a cylindrical structure of resin material is provided further outward, which enables to take out only the wire-like electric wirings 4 to outside without leaking fluid from the portion.

FIG. 2 shows a case of using two heaters to detect an amount of flow and a direction of flow inside the tube. Depending on use, however, there is a way to provide one heater and two sensors for detection, each sensor provided on both outer sides of the heater. In this case, although a measurable range of the amount of flow is narrowed, a less amount of flow allows the amount of flow to be measured with high precision.

FIG. 2 also shows a case of using a cylindrical tube. Even in cases with a tube having other shapes (e.g., oval and square cross section), the present sensor can also deform in accordance with an inner wall structure of the tube to be adapted to the inner wall, taking advantage of flexibility of the film substrate 11. Accordingly, it is preferable that the shape of a sensor installed on the inner wall of the tube is changed in accordance with the shape of the tube to be measured.

Next, FIGS. 3A-3D show connecting methods of the thin film wirings of the film substrate used in the implantable miniature flow sensor of the present embodiment and the wire-like electric wirings.

In FIG. 3A, the wire-like electric wirings 4 are inserted first to grooves 41 provided on a positioning jig 40 so that the wire-like electric wirings 4 are secured. For the purpose of preventing the anisotropically conductive film 15 from adhering to the positioning jig 40 at the last connecting step, a resin film 42 is provided underside the wirelike electric wirings 4 at a connecting point.

It is desirable that material of the resin film 42 is determined in accordance with where to apply. Also, insulation coating is removed from part of the wire-like electric wirings 4 at the connecting point so as to electrically connect with the thin film wirings 13 formed on the film substrate 11 via the anisotropically conductive film 15.

In FIG. 3B, the anisotropically conductive film 15 is placed on the wire-like electric wirings 4 at the connecting point.

In FIG. 3C, the thin film wirings 13 provided on the film substrate 11 are positioned such that the thin film wirings 13 are on the anisotropically conductive film side and are electrically conducted with the wire-like electric wirings 4 at the connecting point. Lastly, thermal compression is applied to the connecting point so that the thin film wirings 13 and the wire-like electric wirings 4 are physically and electrically connected. Metal thin film formation and photolithography are used to prepare the heaters 12 and the thin film wirings 12 onto the film substrate 11.

FIG. 3D shows a schematic view of the m flow sensor 10 prepared in the above method.

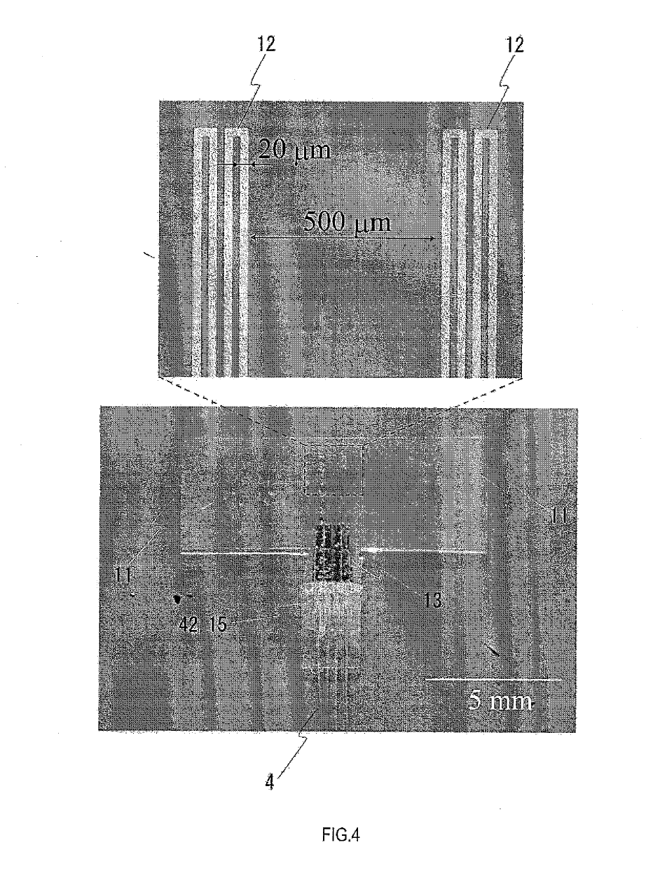

FIG. 4 shows a photo of the film flow sensor of the present invention. An enlarged photo of the heater portion is shown on the upper side in FIG. 4. The overall film flow sensor 10 is shown on the lower side in FIG. 4.

FIG. 4 shows a case in which the heaters 12 and the thin film wirings 13 are formed by chrome (50 nanometers) and gold (250 nanometers) on a resin film having a thickness of a few microns. In FIG. 4, two pairs of heaters are used to detect an amount and a direction of flow inside the tube. However, depending on the intended use, there may be provided one heater and two detection sensors for detection, each sensor provided on both outer sides of the heater. In this case, although a measurable range of the amount of flow is narrowed, a less amount of flow allows the amount of flow to be measured with high precision.

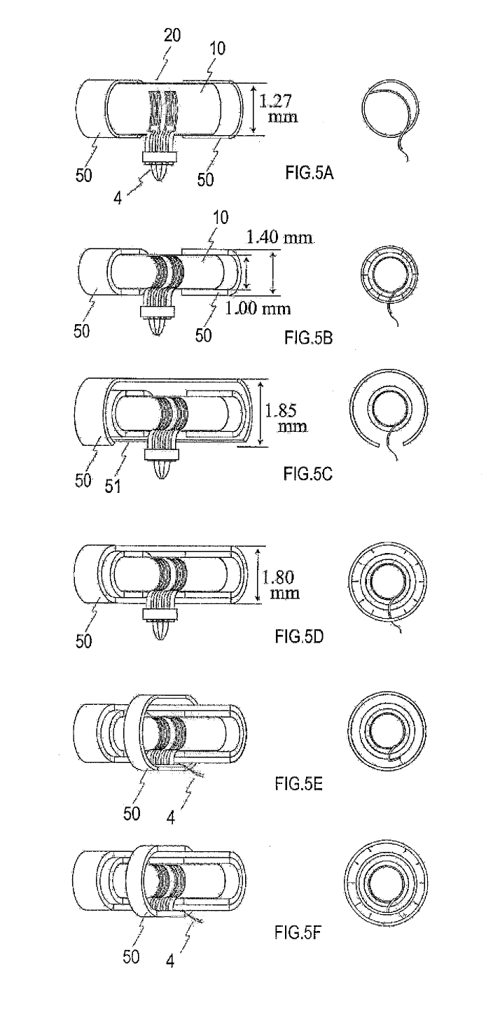

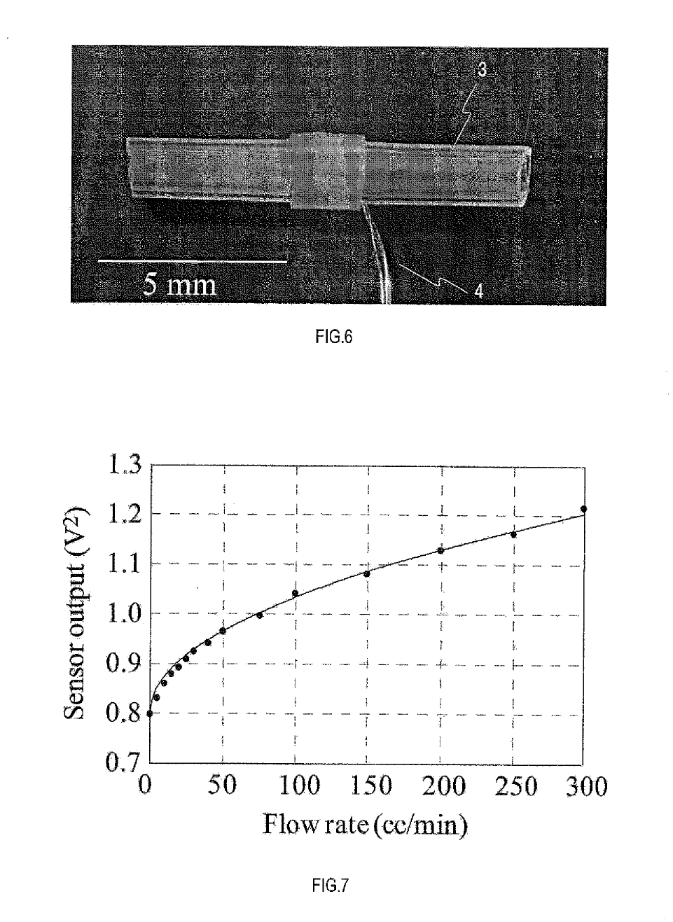

FIGS. 5A-5F show techniques of reducing the size of the implantable miniature flow sensor of the present embodiment to an implantable size of a diameter of a few millimeters or less. In the present embodiment, heat shrinkable resin tubes are used to mount the film flow sensor 10 on the inner wall of the tube. Also, the size of the film flow sensor 10 is designed to be a few millimeters or less. The detail will be described hereinbelow.

In FIG. 5A, heat shrinkable tubes 50 are firstly inserted to both, sides of the heaters 12 of the film flow sensor 10. In this case, the size of the heat shrinkable tubes is relatively large, for example, with an inner diameter of 1.27 millimeters. Thus, the heat shrinkable tubes 50 can be inserted to both sides of the film flow sensor 10.

A cross sectional view taken when the heat shrinkable tubes 50 are inserted to both sides of the film flow sensor 10 is shown on the right side in FIG. 5A. In the present technique, it is not necessary for the film flow sensor 10 to follow the inner wall of the tube upon insertion. It is only necessary to simply insert the heat shrinkable tubes 50 to both sides of the heaters 10 of the film flow sensor 10 for easy operation. The heat shrinkable tubes 50 are not provided at the portion of the heaters 12. As a result, this portion eventually forms a heat insulating cavity structure 20.

Also, the wire-like electric wirings 4 provided in the file flow sensor 10 are designed to be taken outside from between the heat shrinkable tubes 50. It is preferable that the size and material of the heat shrinkable tubes, and the size of the flow sensor, are arbitrarily determined in accordance with conditions for use.

In FIG. 5B, heat is added to the heat shrinkable tubes 50 provided on both sides of the heaters 12 of the film flow sensor 10, thereby to shrink the heat shrinkable tubes 50 to a desired size. At this point, together with the shrinking of the heat shrinkable tubes 50, both end portions of the film flow sensor 10 are automatically mounted to follow the inner walls of the tubes.

in FIG. 5C, again a heat shrinkable tube 50 is prepared, into which the heat shrinkable flow sensor tubes formed at the previous step are inserted. The newly prepared heat shrinkable tube 50 differs from the previously formed tubes in that a slit 51 for taking the electric wirings outside is provided at part of the heat shrinkable tube 50.

In FIG. 5D, again heat is added to shrink the outer heat shrinkable tube 50. At the same time, the outer heat shrinkable tube 50 adheres to the inner tubes to form the heat insulating cavity structure 20. Also, in the process of shrinking the tube, the slit 51 is closed with a film portion for electric wiring being tucked.

In FIG. 5E, for the purpose of physically protecting a connecting portion between the thin film wirings 12 and the wire-like electric wirings 4 by the anisotropically conductive film 15, again a heat shrinkable tube 50 is prepared which can cover only the connecting portion. The heat shrinkable flow sensor tube formed in the previous step is inserted to the prepared heat shrinkable tube 50.

In FIG. 5F, again heat is added to shrink the heat shrinkable tube for wiring connection protection. The heat shrinkable tube adheres to the inner tube so that only the wire-like electric wirings 4 are exposed from the heat shrinkable flow sensor tube.

By employing a process as described above, the two pairs of heaters 12 and the thin film wirings 13 provided on the flexible substrate 11 are identical in shapes. Also, the thin film wirings 13 are taken outside from inside the tube perpendicularly to flow of the fluid flowing through the flow passage 21. Even if the direction of flow is reversed, the two pairs of heaters 12 and the thin film wirings 13 will be in a state reversed under identical conditions. Thereby, regardless of the direction of flow, sensor output characteristics with respect to an amount of flow are the same. As a result, a reciprocating current such as exhalation and inhalation can be measured accurately.

Also, the connecting point between the thin film wirings 12 and the wire-like electric wirings 4 by the anisotropically conductive film 15 is protected by the heat shrinkable tube 50. Only the wire-like electric wirings 4 are exposed from the heat shrinkable flow sensor tube. Even if the implantable miniature flow sensor 3 is implanted in an air passage of an animal, the wire-like electric wirings 4 can be easily drawn to a back portion of the animal under the skin. Also, even after the wire-like electric wirings 4 are taken out from the back portion, wirings can be taken out highly reliably with respect to mechanical load.

FIG. 6 shows a mounting state of the implantable miniature flow sensor of the present embodiment. As explained above, only the wire-like electric wirings 4 are exposed from the heat shrinkable flow sensor tube, so that the flow sensor can be implanted in an air passage of an animal easily.

Now, FIG. 7 shows an example of a relationship between input power and a detected amount of flow of the flow sensor according to the present embodiment. In FIG. 7, the input power to a sensor bridge circuit is shown in relation to the amount of flow when a heater element itself is used as the flow sensor. As shown in FIG. 7, the input power varies depending on the amount of flow. The present sensor can calculate the unknown amount of flow by applying the variation of the input power to a calibration curve.

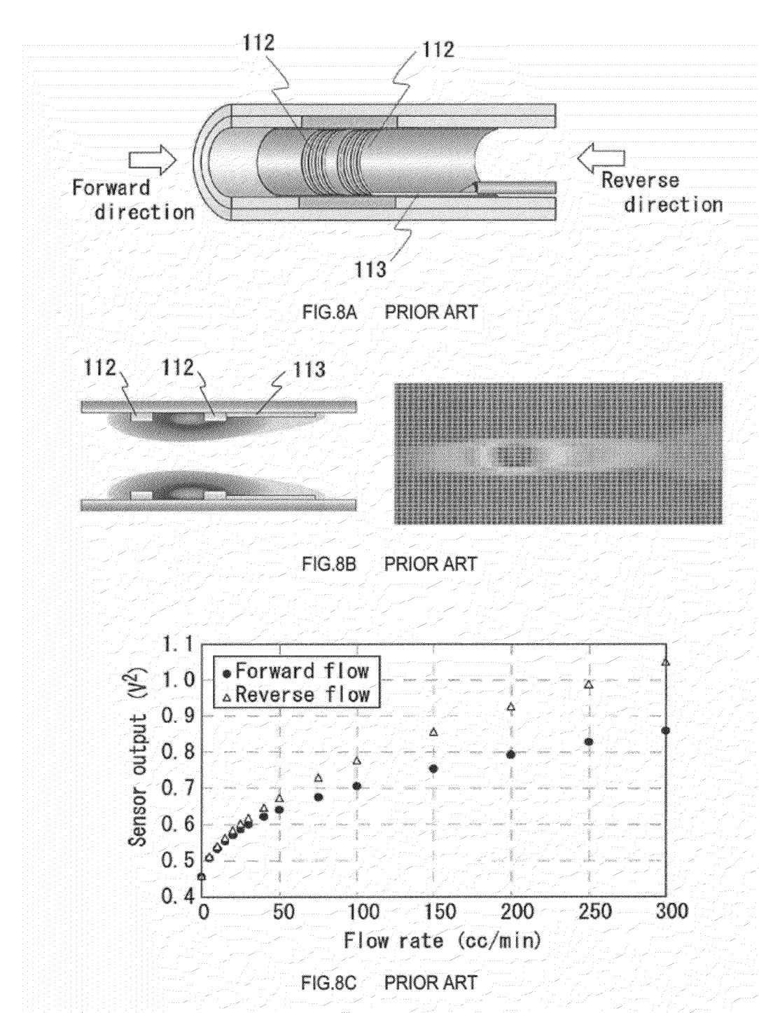

Here, as a comparative example, an electric wiring taken-out structure in a flow sensor of a conventional form (conventional sensor), temperature distribution around heaters, and a relationship between an amount of flow and sensor output are shown in FIGS. 8A-8C. FIG. 8A is a view showing the electric wiring taken-out structure in the conventional sensor. FIG. 8B is a conceptual diagram of temperature distribution around the heaters and a diagram showing a measurement result. FIG. 8C is a diagram showing the relationship between the amount of flow and the sensor output.

As shown in FIG. 8A, in the conventional form, thin film wirings 113 which supply electric power to heaters 112 are configured to be taken out from one direction of a tube inside the tube. Therefore, the thin film wirings 113 also become heating elements. Heat distribution on the heaters is asymmetrical, as shown in FIG. 8B.

As a result, as shown in FIG. 8C, sensor output values differ, depending on a direction of fluid flowing inside the tube. This is because the thin film wirings 113 which supply electric power to the heaters 112 are electrically resistant and thus marginally become heating elements. As the thin film wirings 113 are taken out from the direction of flow of the fluid inside the tube with respect to the heaters 112 as above, heat distribution on the heaters 112 becomes asymmetrical due to heat generation of the thin film wirings 113. As a result, sensor output characteristics vary depending on the direction of the fluid flowing inside the tube.

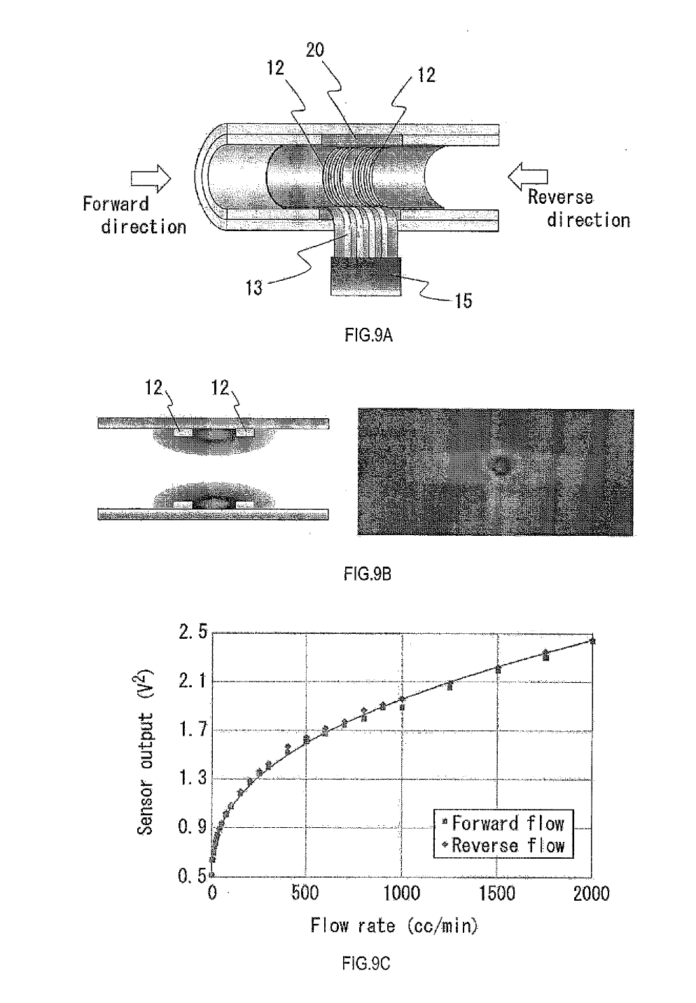

In contrast, the flow sensor of the present embodiment is configured to form thin film wirings that solve the above problem. FIGS. 9A-9C show an electric wiring taken-out structure in the flow sensor of the present embodiment, temperature distribution around the heaters, and relationship between an amount of flow and sensor output. FIG. 9A is a diagram showing the electric wiring taken-out structure in the flow sensor of the present embodiment. FIG. 9B is a diagram showing a conceptual diagram of temperature distribution around the heaters and a diagram showing a measurement result. FIG. 9C is a diagram showing a relationship between an amount of flow and sensor output.

As shown in FIG. 9A, in the present embodiment, the two pairs of the heaters 12 and the thin film wirings 13 are identical in shapes, and form symmetrical structures with identical heating capabilities under identical conditions. Thus, heat distribution by the heaters 12 inside the tube is symmetrical. Further, the thin film wirings 13 are taken out from inside the tube to outside so as to intersect with flow of the fluid flowing through the flow passage,

Thereby, as shown in FIG, 9B, heat distribution on the heaters is symmetrical. As a result, as shown in FIG. 9C, regardless of the direction of the fluid flowing inside the tube, the sensor output characteristics with respect to an amount of flow is the same. A reciprocating current of the fluid flowing inside the tube can be accurately measured,

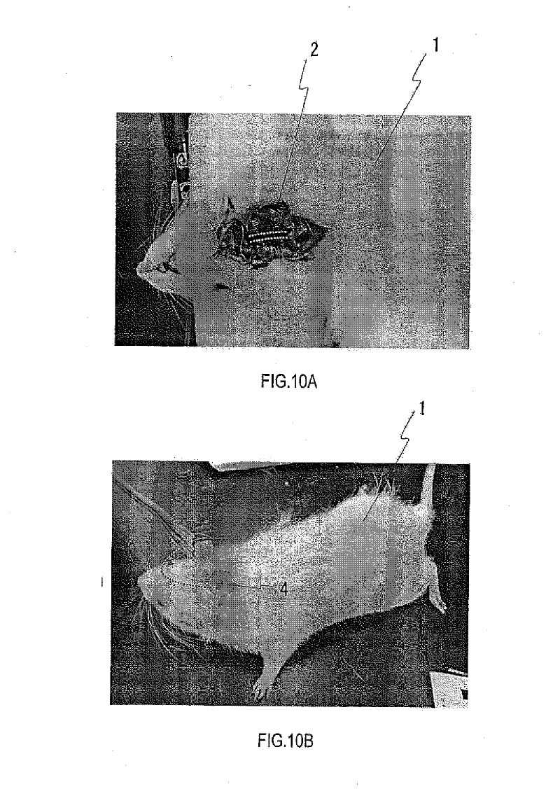

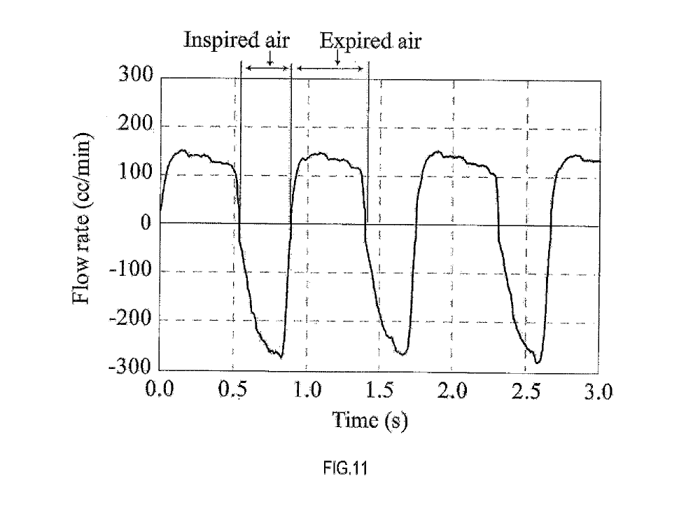

FIGS. 10A-10B show mounting states of the implantable miniature flow sensor of the present embodiment implanted in a rat as an experimental animal.

FIG. 10A shows a state in which the implantable miniature flow sensor of the present invention is implanted in an air passage of the rat.

FIG, 10B shows a state in which, after the implantable miniature flow sensor is implanted in the air passage of the rat, the wire-like electric wirings 4 are drawn to a back side of the head region of the rat under the skin, and thereafter taken outside at a back portion of the rat.

FIG. 11 shows a result at the time when, after the flow sensor was implanted in the air passage of the rat, exhalation and inhalation characteristics at the air passage during activity of the rat were directly examined by the miniature flow sensor of the present embodiment. The result shows that the implantable miniature flow sensor 3 of the present embodiment can actually quantitatively evaluate exhalation and inhalation characteristics at the air passage during activity of the rat.

Matters described in the above FIGS. 1 to 7 are merely examples of the present flow sensor. It is preferable to vary the particular specification in accordance with intended use. Also, the fluid used in the present sensor is mainly gas. The present sensor can be applied to any types of gas. The present sensor can be applied to liquid such as blood and urine, and solid and fluid matters.

As such, in the present embodiment, the two pairs of the heaters 12 and the thin film wirings 13 formed on the film substrate 11 are identical in shapes. Also, the thin film wirings 13 are taken out from inside the tube to outside perpendicularly to flow of the liquid flowing through the flow passage 21. Even if the direction of flow is reversed, the two pairs of heaters and the thin film wirings 13 are in a state reversed under identical conditions. Thereby, regardless of the direction of flow of the fluid inside the tube, the sensor output characteristics with respect to an amount of flow of the fluid are same.

Also, the wirelike electric wirings 4 are connected to the ends of the thin film wirings 13 provided on the film substrate 11 by means of the anisotropically conductive film 15. Thereby, even if the implantable miniature flow sensor 3 is implanted in an air passage of a rat (small animal), the wire-like electric wirings 4 can be easily drawn to a back portion under the skin. Also, even after the wire-like electric wirings 4 are taken out from the back portion, wirings can be taken out with high reliability. As a result, the wire-like electric wirings 4 are designed to be taken out from the back portion of the rat to outside the rat, after drawn to the back portion of the rat.

Also, the film substrate 11 is disposed so as to folio the inner wall of the tube 30. Thereby, turbulence of flow inside the tube due to sensor installment is reduced to the ultimate limit.

### Other Embodiment

In the above embodiment, electric power is supplied to the heaters 12 via the thin film wirings 13 by the wire-like electric wirings from outside the rat. The wire-like electric wirings may be used as antennas, and a microwave receiver may be attached onto the thin film wirings 13.

Then, microwave is externally supplied and received by the wire-like electric wirings as antennas, converted to electric power by the microwave receiver to be supplied to the heaters 12 via the thin film wirings 13. In this case, transmission and reception of signals to and from outside an organism are permitted by the microwave receiver and the wire-like electric wirings used as antennas. With this configuration, operational sections like hands and legs of the organism are unlikely to be brought into contact with the wire-like electric wirings.

1. An in vivo flow sensor that measures an amount of flow of a medium flowing inside a tubular organ of an organism by mounting a flexible substrate, upon which heaters are formed, inside a tube that is disposed in a manner so as to follow the tubular organ of the organism through which the medium is flowing, and by detecting a state of heat quantity generated from the heaters on the mounted flexible substrate being transmitted to the medium,
at least the two heaters formed on the flexible substrate respectively forming symmetrical structures with identical heating capabilities under identical conditions, and linear members, which supply power to the heaters from outside the organism, forming symmetrical structures with identical heating capabilities under identical conditions, thereby allowing heat distribution by the heaters and the linear members inside the tube to be symmetrical, and
the plurality of linear members protrude in a manner so as to intersect from inside the tubular organ to outside the organism.

2. The in vivo flow sensor according to claim 1,
wherein portions of the linear member protruding to outside the organism are at positions where it is difficult for operational sections of the organism to come into contact.

3. The in vivo flow sensor according to claim 2,
wherein a position where the in vivo flow sensor is installed is in an air passage of an animal, and
electric wiring members connected to the linear members taken outside of the animal from a back of the animal.
